(19) United States
(12) Patent Application Publication
(10) Pub. No.: US 2023/0073429 A1
(43) Pub. Date: Mar. 9, 2023

(54) METHODS TO CREATE STRUCTURES WITH ENGINEERED INTERNAL FEATURES, PORES, AND/OR CONNECTED CHANNELS UTILIZING COLD SPRAY PARTICLE DEPOSITION

(71) Applicants: UNIVERSITY OF PITTSBURGH - OF THE COMMONWEALTH SYSTEM OF HIGHER EDUCATION, PITTSBURGH, PA (US); CONCURRENT TECHNOLOGIES CORPORATION, Johnstown, PA (US)

(72) Inventors: Prashant N. KUMTA, Pittsburgh, PA (US); Abhijit ROY, Pittsburgh, PA (US); Howard Arthur KUHN, Butler, PA (US); Matthew CRIADO, Latrobe, PA (US); John Michael OHODNICKI, East Pittsburgh, PA (US); Brock T. GOLESICH, Johnstown, PA (US); Juan J. VALENCIA, Montclair, NJ (US); Michael John POLLOCK, Boswell, PA (US); Michael L. TIMS, Barboursville, VA (US)

(73) Assignees: UNIVERSITY OF PITTSBURGH - OF THE COMMONWEALTH SYSTEM OF HIGHER EDUCATION, PITTSBURGH, PA (US); CONCURRENT TECHNOLOGIES CORPORATION, Johnstown, PA (US)

(21) Appl. No.: 17/795,383

(22) PCT Filed: Feb. 26, 2021
(86) PCT No.: PCT/US2021/019867

§ 371 Date: Jul. 26, 2022

Related U.S. Application Data
(60) Provisional application No. 62/982,782, filed on Feb. 28, 2020.

(51) Int. Cl. B22F 10/25; B22F 10/66; B22F 12/41; B22F 12/53; B22F 10/38; B33Y 10/00; B33Y 30/00; A61L 27/56

(52) U.S. Cl. CPC B22F 10/25; B22F 10/66; B22F 12/41; B22F 12/53; B22F 10/38; B33Y 10/00; B33Y 30/00; A61L 27/56; B33Y 80/00

(57) ABSTRACT

The invention relates to a device and method for preparing a structure or object using an additive manufacturing process referred to as sequential cold spray laser sintering. The method includes depositing by cold spraying a plurality of sequential layers of material onto a substrate/build plate or particles of materials onto a compacted powder bed of material and employing an energy source to sinter or melt each of the plurality of sequential layers or powders to produce sequential sintered layers, wherein the number of additional layers is determined based on those needed to produce the final structure.

### CROSS-REFERENCE TO RELATED APPLICATIONS

This application claims priority under 35 U.S.C. § 119(e) from U.S. provisional patent application No. 62/982,728, entitled “METHODS TO CREATE STRUCTURES WITH ENGINEERED INTERNAL FEATURES, PORES, AND/OR CONNECTED CHANNELS UTILIZING COLD SPRAY PARTICLE DEPOSITION” and filed on Feb. 28, 2020, the contents of which are incorporated herein by reference.

### GOVERNMENT SUPPORT

This invention was made with government support under grant 0812348 awarded by the National Science Foundation (NSF). The government has certain rights in the invention.

### FIELD OF THE INVENTION

The invention relates to systems and methods to create structures with engineered internal features, pores, and/or connected channels utilizing additive manufacturing and, more particularly, preparing structures including geometrically complex structures using a novel sequential cold spray laser sintering approach.

### BACKGROUND

Additive manufacturing (AM), commonly referred to as 3D printing, is a process wherein a three-dimensional (3D) object is created by layered addition of material in predetermined locations over many layers. Each layer of material is fused with the underlying layer utilizing either thermal, electron beam, or laser energy to facilitate particle melting or sintering. Relying on either melting or sintering allows the use of many material types as feedstock particles, including numerous variants of conducting, insulating, or semiconducting plastics, ceramics, and metals. Pairing this ever-increasing selection of compatible materials with the different building methods provides a plethora of attractive and beneficial attributes not available with any other manufacturing process. The benefits of AM are numerous due to the uniqueness of these methods for creating myriad complex parts, including generation of customized objects driven by optimization overcoming the limitations of conventional manufacturing methods. AM technology has therefore been applied to many manufacturing sectors offering increasing efficiencies, generation of low volume and highly complex parts with reduced materials consumption and reduction in production cost. AM is thus rapidly evolving as the foundation platform for many future manufacturing technologies with seamless integration into many research areas.

Of the various types of AM, the metal-based processes provide the most benefits for generation of not only prototypes but also functional parts of near and fully net-shaped objects. The current methods of metal AM classifications include Selective Laser Melting (SLM), Selective Laser Sintering (SLS), Laser Engineered Net Shaping (LENS), high powered electron beams (E-Beam), Binder Jetting (BJ), and several other lesser known variations primarily used for specific materials or custom builds. These aforementioned metal AM processes differ in the following aspects: (i) Feedstock (powdered metal versus metal wire filaments); (ii) The methods of material joining or fusing (melting, sintering, or a primary chemical binder followed by a secondary post AM process sintering phase); and (iii) Extent of the amount of post AM processing required for completion of a part. Despite many options, the SLM and the BJ processes are the most commonly used metal powder-bed based AM methods. The popularity of these AM methods stems from process resolution and precision, ease of use, and applicability for a wide range of acceptable materials. Each of these two methods use different approaches to fuse the metal particles while creating the desired 3D object. In the case of SLM, the powder is melted with a high intensity laser traversing over selected areas of each layer within the powder-bed. With binder jetting, on the other hand, the particles within the powder bed are bound together during the printing process using a binder over selected areas of each layer. The process requires post process sintering to eliminate the binder and create a strong metallic bond between the metal particles.

The limitations of the above metal powder-based feedstock methods are categorized into two general areas: (i) The feedstock material requirements and (ii) The location-setup-environment of the AM apparatus. In the feedstock material selection, it is crucial to select a material that is non-reactive and oxide free or has an oxide decomposition temperature much lower than that of the intrinsic melting temperature of the material. Furthermore, highly reactive metals such as aluminum (Al) and titanium (Ti) require tightly controlled inert environments within the printer to make the process amenable and safe. In the case of magnesium (Mg), however, the inert environment needs to be held at an elevated overpressure to compensate for the high vapor pressure of the material as well adding yet another level of complexity over and above the safety concerns posed by the handling of the reactive fine powder itself. Even with all the precautions observed, attempts to additively manufacture Mg results in a final structure that is insufficient for any industrial uses due to the mechanically weak structure caused by the high porosity and poor propensity for achieving sintering to full density. The porosity issue is not exclusive to Mg alone and this issue is commonly found with many other materials, which have undergone any one of the several AM processes where high temperatures are observed.

The second area where the powder-based feedstock AM processes requires care to prevent poor quality prints is linked to the operational environment and setup of the overall AM apparatus. To prevent mid-process failures, and maintaining high resolution while allowing homogeneous powder distribution for each additional layer, the AM printer needs to be correctly orientated. For the best viable outcome, it is essential that the printer is perfectly level, free from external vibrations and high volume air flows while being operated within the correct external temperature and humidity range, placing the printer in an environment where gravity is the essential and primary dominant force limiting its applicability under microgravity and low earth orbit environments. Each of these factors have the propensity for negatively affecting the placement and retention of the micron-sized metal particles until they can be fused to the larger print. As a result, artifacts and voids are commonplace with the printing process combined with suboptimal printer conditions. Moreover, failed prints may lead to printer damage, which is a major limitation. Due to these elusive requirements, the AM apparatuses are not robust enough to be easily moved or kept on a mobile platform for onsite parts production during a repair at a remote location.

Thus, there is a need and desire in the art to develop new AM systems and methods that compensate for all of the above factors relating to material selection, and provide a more robust printing platform. Moreover, there is a need for a versatile AM system that can offer generation of complex structures of full density without the problems associated with having a gravity dependent fixed powder bed aside from the other problems of vapor pressure and oxide layers akin to reactive metals.

### SUMMARY OF THE INVENTION

In one aspect, the invention provides a method of preparing a multi-layer structure. The method includes: a) cold spraying a layer of feedstock material onto a substrate/build plate to create a cold sprayed coating; b) employing an energy source to sinter or melt the cold sprayed coating, including directing the energy source to conduct localized sintering or melting of specific particles or regions of the cold sprayed coating that correspond to a preselected multi-layer structure; and forming a sintered or melted layer; and c) sequentially repeating the steps of a) and b) forming one or more additional material layers on the first material layer, wherein the number of additional layers is determined based on a total number of layers needed to produce the preselected multi-layer structure.

In certain embodiments, cold spraying is conducted in large passes over the entire substrate/build plate, or in precisely located areas with a tight spray pattern to reduce the amount of material usage. In certain embodiments, cold spraying is conducted at variable speeds to generate compact powder beds with particles in contact or in precisely located areas within a tight spray pattern to reduce the amount of material used.

In certain embodiments, the feedstock material is in a dry form selected from the group consisting of particles, particulates, or granules.

In certain embodiments, the sintering or melting process results in a fragmented oxide dispersed within the sintered or melted material and the preselected multi-layer structure. The dispersed fragmented oxide can serve as an oxide filler imparting an oxide dispersion-strength to the preselected multi-layer structure.

In certain embodiments, adding a second material layer to the first material layer is effective to control or eliminate porosity in the first material layer.

In certain embodiments, incoming particles impinge and peen the first layer causing collapse of pores induced by sintering or fusion of the first layer.

In certain embodiments, following completion of the first material layer and a subsequent second material layer, power of the energy source is increased and used in trimming and shaping an outline of the preselected structure being prepared. Following the trimming and shaping, one or more additional material layers can be formed until the preselected multi-layer structure is built in its entirety with the corresponding complexity, contiguity, shape, and size.

In certain embodiments, each of the feedstock materials comprises a material selected from the group consisting of metal, metal alloy, polymer, ceramic, semiconductor, and mixtures and combinations thereof. The metal or metal alloy can be selected from the group consisting of magnesium, copper, aluminum, stainless steel, tungsten, titanium, tantalum, Inconel, rhenium, tungsten-niobium alloy, tungsten-cobalt alloy, tungsten-rhenium alloy, tungsten-silver composite, tungsten-tungsten carbide composite, and mixtures or alloys thereof.

In certain embodiments, the method includes employing a CAD model corresponding to the preselected structure for directing and controlling the energy source in step b). Step b) can form pores and/or pathways in the preselected multi-layer structure.

In certain embodiments, the method further includes, following step b), subjecting the sintered or melted layer to subtractive machining, which includes smoothing the surface of the sintered or melted layer; and forming a first material layer of the preselected multi-layer structure.

In certain embodiments, the energy source is selected from the group consisting of laser, electron beam, electric arc, induction, magneto resistance, electrical resistance, and combinations thereof.

In another aspect, the invention provides an additive manufacture device, including a cold spray apparatus to deposit a coating of feedstock material on a substrate/build plate; an energy source to sinter or melt the coating of feedstock material to form a sintered or melted layer; and a CAD model to direct the energy source to sinter specific particles or regions of the coating of the feedstock material that correspond to a preselected multi-layer structure, wherein one or more additional layers of feedstock material are sequentially deposited on the first material layer to produce one or more additional material layers, and wherein the number of additional material layers is determined based on those needed to produce the preselected multi-layer structure.

In still another aspect, the invention provides an additive manufacture device, including a cold spray apparatus to deposit compact powder of feedstock material on a powder bed generating a compact powder bed; an energy source to sinter or melt the compact powder bed of feedstock material to form a sintered or melted layer; and a CAD model to direct the energy source to sinter specific particles or regions of the compact powder layer of the feedstock material that correspond to a preselected multi-layer structure, wherein one or more additional layers of feedstock material are sequentially deposited on the first material layer to produce one or more additional material layers, and wherein the number of additional material layers is determined based on those needed to produce the preselected multi-layer structure.

In certain embodiments, the feedstock material includes a material selected from the group consisting of metal, metal alloy, polymer, ceramic, semiconductor, and mixtures and combinations thereof.

In certain embodiments, the first material layer and one or more additional layers comprise pores and/or pathways.

In certain embodiments, the multi-layer structure is a medical implant device. The medical implant device can be a scaffold. The medical implant device can be composed of a material selected from the group consisting of Mg, Mg alloy, and Mg composite.

In certain embodiments, each of the one or more additional material layers is composed of the same feedstock material. In certain other embodiments, one or more of the additional material layers of the multi-layer structure is/are composed of a different feedstock material as compared to the other material layers.

In certain embodiments, the feedstock material for each material layer is randomly selected. In certain other embodiments, the feedstock material is selected such that alternating material layers are composed of the same feedstock material or a pattern of material layers is composed of the same feedstock material.

In certain embodiments, the device further includes a subtractive machining apparatus to smooth the surface of the sintered or melted layer and form a first material layer of the preselected multi-layer structure.

### BRIEF DESCRIPTION OF THE DRAWINGS

A full understanding of the disclosed concept can be gained from the following description of the preferred embodiments when read in conjunction with the accompanying drawings, as follows:

FIG. 1 is a schematic that shows the steps of a sequential cold spray laser sintering (SCSLS) process, in accordance with certain embodiments of the invention;

FIG. 2 is an image that shows a cold spray magnesium sample sprayed on substrate, in accordance with certain embodiments of the invention;

FIG. 3 is an image that shows a cold spray magnesium cross section depicting post spray before the sintering procedure wherein the substrate interaction is visible near the bottom, in accordance with certain embodiments of the invention;

FIG. 4 is an image that shows a cold spray magnesium cross section depicting a reduction in mechanical interlocking, in accordance with certain embodiments of the invention;

FIG. 5 is an image that shows a cold spray magnesium cross section depicting a mixture of mechanical and metallic bonding, in accordance with certain embodiments of the invention;

FIG. 6 is an image that shows a cold spray magnesium cross section depicting full metallic bonding, in accordance with certain embodiments of the invention;

FIG. 7 is an image that shows a cold spray magnesium cross section depicting a solid structure with least amount of porosity, in accordance with certain embodiments of the invention;

FIG. 8 is an image that shows a cold spray magnesium cross section depicting a mixture or combination of closed pores and interconnected channels, in accordance with certain embodiments of the invention;

FIG. 9 is an image that shows a cold spray magnesium cross section depicting a close-up of an interconnected channel as shown in FIG. 8, in accordance with certain embodiments of the invention;

FIG. 10 is an image that shows a cold spray magnesium cross section depicting the creation of areas of pores/channels and solid areas in a single sample, in accordance with certain embodiments of the invention;

FIG. 11 is an image that shows elemental surface mapping of the sample in FIG. 3 (having an Al substrate), in accordance with certain embodiments of the invention;

FIG. 12 is an image that shows elemental surface mapping of the sample in FIG. 4, in accordance with certain embodiments of the invention; and

FIG. 13 is an image that shows elemental surface mapping of the sample in FIG. 8, in accordance with certain embodiments of the invention.

### DETAILED DESCRIPTION OF THE PREFERRED EMBODIMENTS

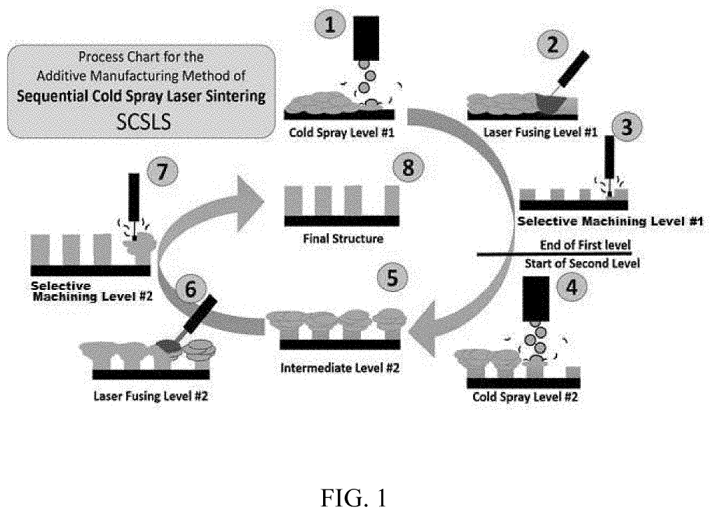

The invention relates to systems and methods to create structures with engineered internal features, pores, and/or connected channels utilizing additive manufacturing and, more particularly, preparing structures including geometrically complex structures using a novel sequential cold spray laser sintering (SCSLS) process. The SCSLS process is a multistep additive manufacturing process comprised of components or procedures that mimic cold spray (CS), selective laser melting (SLM), and selective laser sintering (SLS) all in tandem or either in full or in part. Further, the invention relates to an additive manufacture printer or device in which cold spray is used as a method to deposit the next layer amount of material to advance the print while a laser sinters the cold spray deposition to coalesce the particles creating the SCSLS approach. Additionally, the approach affords the ability to control the speed of the cold spray process enabling cold spray deposition of particles at variable speeds ranging from (˜0.01 m/s to 11,000 m/s) thus resulting in a sequential variable speed cold spray laser sintering (SVSCSLS). Finally, the process also affords the use of the selective laser to allow for selective subtractive machining leading to controlled surface finish of the construct after every layer is deposited following CS and LS or laser melting (LM) as depicted in FIG. 1.

The term “laser” is used herein as an exemplary energy source for use in the invention, and it is contemplated and intended in accordance with the various embodiments of the invention that alternative energy sources are essentially suitable for use, such as but not limited to, electron beam, electric arc, induction, magneto resistance, electrical resistance, and combinations thereof.

In certain embodiments, the invention provides systems and methods for producing biomedical bone implants, e.g., scaffolds, for regenerative medicine using magnesium (Mg) and/or Mg alloys. The implants prepared according to the invention meet mechanical in-vitro corrosion and cytotoxicity properties, provide a surface finish that is effective (e.g., optimized) for bone growth, and have designed/engineered pores that allow a path for cells to receive nutrients and start bone regeneration from multiple sites within the implant.

The systems and methods of the invention include deposition of a feedstock material on a surface or substrate (e.g., build plate) using a CS deposition apparatus and techniques to form a coating or layer. The feedstock material includes metal or metal alloy, ceramic, or polymer, or mixtures or combinations or composites thereof. Suitable forms of the feedstock material include a dry form, such as particles, particulates, or granules. In certain embodiments, the material is Mg particles or Mg alloy particles. The CS deposited coating or layer is then subjected to localized laser sintering (LS) or laser melting (LM) to coalesce and fuse the deposited particles. The laser is directed and/or controlled by a control file, such as but not limited to Computer Aided Drafting (CAD) model/software, that corresponds to the desired or selected multilayer structure that is to be formed. The sintered or melted CS coating or layer is then subjected to subtractive machining (SM) to smooth the surface of the coating or layer, which also contributes to corrosion protection. This process allows one layer of the multi-layer structure to be completed. Additional layers are then sequentially added to this first layer or base layer. The forgoing steps are then repeated for the sequential deposition of additional layers to form, e.g., build, the multi-layer structure. Each of the additional layers include a feedstock material, e.g., metal or metal alloy, ceramic, or polymer, or mixtures or combinations thereof, that is the same or different than the feedstock material of the first layer. In certain embodiments, each of the layers of the multi-layer structure is composed of the same feedstock material. In other embodiments, one or more of the layers of the multi-layer structure is/are composed of a different feedstock material as compared to the other layers. In certain embodiments, the feedstock material for each layer is randomly selected, in other embodiments, the feedstock material is selected such that alternating layers are composed of the same feedstock material or a pattern of layers are composed of the same feedstock material.

The invention includes novel developed additive manufacturing (AM) systems and methods that provide a more robust printing platform. The new AM methods are effective to achieve a robust structure with mechanical properties that match the following specifications, e.g., for tungsten (W) and other refractory metals and metal alloys.

TABLE 1

| Properties | Values |
|---|---|
| Young’s Modulus (@100° C.)(GPa) | >340 |
| Vickers Hardness (@100° C.)(MPa) | >3500 |
| Ultimate Tensile Strength (@2000° C.) | >70 |
| (MPa) | |
| Yield Strength (@2000° C.)(MPa) | >50 |
| Total Porosity (%) | <10 |
| Surface Roughness (μm) | <3.0 |

The SCSLS approach is a manufacturing process combining CS and SLS or SLM benefits to create parts with desired or engineered characteristics or properties, e.g., minimum porosity and micro-cracking, as compared to other AM systems and methods for refractory metals and their alloys. The process also produces application-specific 3D parts with excellent shape, size, pore geometry, and surface control. The CS process creates a mechanical bond between particles. This interaction is facilitated by extensive particle deformation creating a dense layer offering more material contact and thus, mass to transmit thermal energy.

As used herein relating to the SCSLS process, the term “layer-by-layer” or “layer” and like terms do not limit the process from working solely on a two-dimensional (X-Y) plane where an increase in a layer is a gain only in the Z direction. The term “layer-by-layer” also includes deposition and/or laser angles that may not be perpendicular to the X-Y plane and may include planes based in the Z-Y, Z-X, and any sub-planes within or any planes not orthogonal to X-Y, Z-Y, or Z-X planes.

The term “material(s)”, “feedstock material(s)” and like terms include metallic-, ceramic-, or polymeric-based materials. A wide variety of materials are suitable for use in the SCSLS process, including metals, metal alloys, and metal composites, such as but not limited to magnesium, copper, aluminum, stainless steel, Inconel, tungsten, titanium, molybdenum, tantalum and other refractory metals, and mixtures or alloys or composites thereof, and ceramics, polymers and mixtures, blends, or composites thereof. In certain embodiments the materials include tungsten, tungsten alloys, such as tungsten-titanium, tungsten-tantalum, tungsten-niobium, tungsten-cobalt, and tungsten-molybdenum alloys, and tungsten composites, such as tungsten-copper, tungsten-silver, tungsten-tungsten carbide, and tungsten-rhenium composites. In certain other embodiments, the materials for use in the SCSLS process include polymers, semiconductors, and ceramics pertaining to both the sacrificial support structures and the primary and/or additional materials structures used to make the final structure or sacrificial features needed to ensure a proper final build. Suitable material(s) or feedstock material(s) for use in the invention include a variety of forms. Non-limiting examples include a dry form, such as particles, particulates, or granules.

The term “control file” and like term is a generic term used to define any method to geometrically control any deposition or laser device(s) receiving commands from a structure rendered within a computer, digitalized to linear movements, and sent to the motion control devices to precisely control the build of the model within the AM device. Non-limiting examples of such methods used include but are not limited to CAD software, G-code, STL files, drafting program, slicer program, computer modeler, or digital modeler.

The SCSLS systems and processes additively create three-dimensional (3D) parts or structures from feedstock materials. Current parts made by known sintering- or melting-based AM methods are unsatisfactory, lacking control of pore formation yielding undesired porosity. Materials with intrinsic oxide layers sinter poorly entrapping impurities that create porosity and inclusions within the structure. The mechanical strength is compromised due to the creation of internal micro-cracks and ensuing micro-channels preventing creation of any form of a heretic seal. Pores yield inferior mechanical properties requiring the creation of thick coatings for shielding and wear protection ensuring minimum thickness meeting protection needs. There are great benefits in creating a homogenous, thin, pore- and defect-free coating hence, reducing weight without sacrificing protection.

Additionally, known AM methods fail to create complex parts of technologically viable refractory metals. Further, AM-made parts fail to meet the mechanical strength of parts made by powder metallurgy (PM) mainly due to the large amount of cracks and pores. While PM-created parts are reliable and exhibit acceptable materials properties, the process considerably limits the complexity of the structure including the inability to add many internal features. The dense coating applied by CS most closely matches PM prior to sintering caused by the extreme plastic deformation enabling large particle-particle contact areas. In addition, the dense packing reduces shrinkage during sintering since there are fewer voids allowing the generation of complex structures having low levels of porosity, reduced dimensional drift, and moreover, displaying improved mechanical properties.

The SCSLS process according to the invention is effective to create complex 3D parts of metals, metal alloys, and metal composites, such as magnesium (Mg), tungsten (W), and other germane refractory metals, for use in a wide variety of applications. The SCSLS process is a hybrid AM-subtractive machining (SM) approach embedding current AM-SM technologies into a single process. Thus, the SCSLS process according to the invention utilizes features found in several current AM processes resulting in the hybrid approach that offers a resilient, robust printing method extending its operation in terrestrial as well as micro-gravity environments. The SCSLS process uses the sequential CS deposition approach for adding material to the system's build plate followed by a modified SLS or SLM process to coalesce the powder particles into a homogenous structure, while also controlling, e.g., reducing or eliminating, voids, and porosity with the aim of realizing near-bulk material mechanical properties with no print quality relationships attached to the surrounding environment.

Since the SCSLS process combines AM and SM processing, the initially rough finish of a CS-laser fused surface can easily be corrected in operando during the formation of each layer which is not possible with other AM methods including BJ, SLM, and SLS. Only parts made by the directed energy deposition (DED) process can be machined in-situ while the other AM processes require ex-situ additional post processing (grinding/machining) to achieve a smooth surface finish. Conventional SM is conducted utilizing either a monolithic torus cubic boron nitride cutter (Rₐ>0.5 μm) or laser cutting/polishing (Rₐ>2.5 μm) methods. These roughness values are translatable to the SCSLS processes since the process is designed to be created as a set of tool heads for use within a computer numerical controlled (CNC) mill or lathe. External surface finishing is necessary with AM-created parts, but having the ability to add a smooth finish in operando to internal passages as the structure is being created in SCSLS offers vast opportunities with the optimization of fluid flows relating to the preservation of laminar flow. Additional SM benefits to the SCSLS process include the creation of features rendered difficult to create via conventional AM processes such as threaded mounting holes and high-precision lateral circular passageways, as well as other features.

Further, unlike sintering by PM, in the SLM process the laser creates and maintains a melt pool to combine the feedstock powders. During this melting phase, the high surface tension of the melt pool draws in the adjacent material particles. Accommodation of larger volume and changing depth of the melt pool allows different fluid dynamics and temperature gradients to exist causing many unfused particles and voids to remain below the melt. Furthermore, the high melting temperature of the material is well above the disassociation temperature of the oxide layer causing the creation of gaseous voids. Additional pores are thus created by the voids left between the loosely packed powder (50-74% packing factor) with even more being created by the unfavorable SLM parameters resulting in balling and keyhole phenomenon randomly dispersing undesired pores throughout the structure. In contrast, SCSLS provides improved particle fusion since the laser immediately interacts with a layer of particles generated by CS held tightly in a dense oxide-free CS deposited layer. Due to the nearly homogeneous CS layers, once the melt pool is formed, the surface tension cannot consume adjacent feedstock thus, maintaining a constant volume within the melt pool. The reduction in oxide volume within the dense CS-deposited layer also decreases the liberation of gaseous bubbles and hence, further eliminates creation of interlayer voids giving pore-free parts. The oxide particles are also either ejected from the surface of the CS layer or embedded and dispersed within the melt pool created by the LM step in the SCSLS process thus resulting in an oxide dispersion strengthened structure leading to further enhancement in strength and other related and characteristic properties of the material. Furthermore, control of the cooling rate due to the laser melting can also result in metastable microstructures of fine grains or even stabilization of non-equilibrium phases including amorphous layers resulting in unique mechanical, electronic, and biological properties.

Micro-cracking is typically observed along the grain boundaries of SLM created material structures due to the liberated gas movement from the oxide layer to the grain boundaries during melt solidification. These voids, which act as stress concentrators paired with the strain associated with rapid melting and cooling of the localized thermal event, causes cracks to form and propagate. These cracks are not normally found in sintered parts due to the differing process dynamics and only have little correlation to the operational parameters of the laser. Whereas in the SCSLS process, the feedstock powder is plastically deformed upon impact within the SCSLS process and the oxide layer is prone to fragmentation and ejection from the process. This causes reduction of the oxide itself, thus mitigating the formation of micro-cracks and generation of pores. With the reduction in cracks as a result of the SCSLS process, a higher densification percent can be attained while yielding a more homogenous structure that will likely display much higher mechanical strength values.

Simultaneous deposition of multiple materials is feasible within the SCSLS processes. For fusing the different materials, laser sintering, melting, or a combination of both may be required at precise locations. In certain embodiments, the platform of a CNC mill allows for adjusting and applying different laser intensities directly to the material to which they correlate while not affecting the other surrounding materials. Issues may arise if dissimilar materials having no solubility in the liquid state and/or solid state or materials having widely varying melting temperatures are placed in close proximity. If this is desirable, in certain embodiments, the materials are built around each other with unique interlocking geometry sealing and then finally sealed with a conventional CS coating for prevention of a thermal expansion mismatch or vibration related loosening.

According to the invention, the process of CS is used because it is unique in that it uses a high-pressure gas to accelerate particles to a critical velocity, allowing particle collision under supersonic conditions with a given surface. The particles collide onto the substrate transferring the kinetic energy created by the heated, high-pressure gas stream traveling at a critical velocity. Once this collision occurs, the energy transmitted into the particle causes colossal plastic deformation of the particles while strongly adhering to the colliding surface. This adherence interaction between the particle and surface creates features such as adiabatic shear instabilities, cold welding, and interlocking due to the flow of the particles during deformation thus ensuring a strong bond (e.g., by firmly adhering the feedstock to the structure). Because of this strong bond, CS is a useful technique for applying strong coatings at low temperatures, relative to the melting point of the materials, onto base materials, thus allowing dissimilar metals to be cladded to one another while still being capable of performing structural repairs. Furthermore, the extreme particle deformation and speed at which the collisions occur causes fracture of the inherent thick oxide layer, particularly in reactive metals leading to violent removal and ejection of the oxide from the intermediate material building area. As this oxide layer removal occurs, the deforming particle simultaneously bonds with the other nearby particles, forming a tight bond between the particles, thus preventing the formation of an additional oxide layer. This oxide-free contact area allows the preservation of a clean metal-metal interaction area, favorable for thermal sintering. The uniqueness of the CS bonding mechanism is also however, a limitation since the cold-sprayed metal exhibits limited ductility and tends to be brittle with an increased hardness rendering the 3D printed part inappropriate for certain applications. Furthermore, the CS process is unable to precisely place particles with high resolution due to the turbulent nature of the accelerant gas paired with the slight deviation of particle sizes and shapes causing a range of velocities and trajectories of the incoming particles. Despite these issues related to CS, large-scale sintering is useful to fuse the CS coatings thus increasing the ductility of the system. To accomplish a more thorough sintering capable of creating complex structures within the CS coating, the AM method of SLS or SLM is utilized, thus completing the SCSLS approach.

The SCSLS process commences with a cold spray apparatus that deposits a layer of material onto a substrate/build plate. This step can be done either in large passes similar to generation of coatings over the entire substrate/build plate, or in precisely located areas with a tighter spray pattern to reduce the amount of material usage. Once the feedstock material is deposited by CS to create the first layer, the sintering laser or melting laser is employed to move over the surface at a set power level and scanning speed resulting in sintering of the underlying immediate CS-deposited particles together, as per the directive of a control file, e.g., a Computer Aided Drafting (CAD) model/software corresponding to the desired structure. The laser sintering process also allows the fragmented oxide to be dispersed within the sintered material's structure serving as an oxide filler creating an oxide-dispersion-strengthened material structure. At the conclusion of this laser interaction with the first layer, the deposition of the second layer by CS is then initiated. In concurrence with the first layer, the CS feedstock powder is applied over the laser-sintered first layer thus forming the second layer. The process is then repeated sequentially. This interaction has the added benefit of eliminating porosity, which results from the incoming particles impinging and peening the first layer thus causing the collapse of any pores induced by laser sintering or laser melting of the first layer. Once the CS step creating the second layer coating is completed, the SLS laser or SLM is once again activated and directed by the control file, e.g., CAD file, as to which particles need to be sintered onto the second layer to coincide with creating the desired or selected structure. After the sintering is completed, a break in the current cycle is performed where the power of the laser is increased and used to trace the outline of the object being manufactured and its interaction with the substrate/build plate. This step facilitates removal of the extra material placed during the CS interaction through vaporization or cutting of the non-sintered particles, such that the forming structure is as precise as possible with the desired surface finish. At the conclusion of this trimming and shaping sequence, the third layer is initiated with a repeat of the same foregoing order of events to deposit the new material and fuse it with the growing structure, repeating on each layer until the desired 3D object is built in its entirety preserving the complexity, contiguity, shape, and size of the part.

In certain embodiments, the SCSLS process is a three-part process, which needs to be repeated per every layer's addition of the print until the entire structure is completed. The steps involved in the three-part process include the following:
Step 1. The process of CS is used with a high-pressure gas into which a powder stream is introduced and allowed to accelerate with the expanding gas until the powder particles reach a critical velocity and are allowed to strike a substrate or build plate. Upon this collision, the particles experience rapid deceleration and transmit the kinetic energy gathered into a massive amount of deformation, which allows the flowing material to interlock onto the substrate or previously placed layer(s) of CS material. The deformation process also allows for fragmentation of the inherent oxide layer in the case of the reactive metals and trapping them within the interlocked deformed layers. This CS coating is the method of powder addition for the AM apparatus of the SCSLS process. Additionally, the CS particles can be transported at controlled velocities in the range of ˜0.01 m/s to 11,000 m/s thus enabling a uniformly contacted powder bed at low velocities without inducing deformation and allowing laser sintering and laser melting or both at much lower laser powers than the conventional SLM process. At very high velocities, the process will depict that of traditional CS with the particles undergoing colossal plastic deformation.
Step 2. With a CS layer tightly adhered to and coating the substrate or build plate, or a compacted powder bed as indicated in Step 1, a laser installs energy into selected areas to promote migration of material components and ensuing diffusion of the atoms or ions to induce particle-particle coalescence resulting in reduction in surface area with corresponding sintering and densification without inducing particle melting. The laser can, however, also induce energy to cause particle fusion and corresponding densification. Taken together, this all-encompassing step converts the physical adhesion methods installed to the system via CS into a metallurgical or metallic bond in the case of metals, a covalent or ionic bond in the case of a ceramic, and a covalent, secondary, or hydrogen-bonded polymeric bond wherein the individual particles coalesce into a single structure. The coalescence occurs without melting in the case of the metal and ceramic powder feedstock or fusion in the case of the polymer powder feedstock while eliminating porosity with the shared grains or polymer particles throughout the entire structure. At the same time, the sintering and fusion process can lead to dispersing the fragmented oxide causing oxide dispersions within the sintered material part correspondingly contributing to strengthening the sintered material via dispersion strengthening mechanisms. The laser is precisely and meticulously moved while being controlled by geometric instructions, e.g., control file, to create a 3D part of the computer-designed rendering of said part. A single pass or several passes over the same area may need to be completed depending on the amount of particle-particle sintering or particle-particle fusion available from the laser. Depending on the material being non-sintered and the possibility of future layer interference, Step 3 may be necessary to remove any unwanted material. Alternatively, the process may return to Step 1 if it is deemed that the unused material is needed or poses a threat to the rest of the building sequence and then following Steps 1 and 2, Step 3 may be necessary.
Step 3. Due to the overspray provided by Step 1 of the CS process, the laser either from Step 2 or a more powerful laser is employed to cut or vaporize the over-sprayed material away from the features of the printed structure. This will allow the designed object, needed support material, or other needed material not for supporting the designed object to remain while the unused/unneeded powder is removed to prevent interference with the ongoing process. After this step, the process is started back at Step 1 signaling the start of a new layer.
Step 3a. This step serves the same purpose as Step 3 in that the removal of the overspray material is necessary. The difference between these two steps is based on this step which describes using conventional machining tools such as cutting, milling, or other processes to replace the laser vaporization/cutting detailed in Step 3. Upon the completion of this step, the process returns to Step 1 to repeat the cycle for the next layer of the build.

With respect to Step 1, the high-pressure gas (accelerating gas) includes a variety of known gases and mixtures, such as but not limited to air, nitrogen, or helium, or mixtures thereof. This gas or mixture thereof is used to assist in optimizing the critical velocity needed to perform the task. The accelerating gas is heated, chilled, or left unaffected to assist with the optimization of the process. The process of Step 1 is designed to provide intrinsic strength between the particles to allow for additive manufacturing to be executed in environments where constant motion leads to movement and detachment of the particles from their respective location. In the inventive process, the particles remain stationary due to the mechanical bonding created by the cold spray process. Therefore, the process is useful in a wide variety of environments, including, for example, in rough waters, in overland/air/water/underground transportation, or when placed on a weak or unstable foundation, random vibrations, and the like. Further, in Step 1, the process is designed to provide intrinsic strength between the particles to allow for AM to be conducted in environments where microgravity/non-earth gravity may allow the particles to move from their location making the current powder beds used in BJ and SLM very tenuous for use under microgravity due to the weak or absence of gravitational forces necessary to keep the powder particles in contact. However, in contrast, in the case of the SCSLS the particles remain stationary due to the mechanical bonding created by the cold spray process or compaction of the powder particles facilitated by the variable speed cold spray process. Such reduced gravity environments include but are not limited to in orbit, on other planets, on other moons, and the like.

In Step 1 of the SCSLS process, as aforementioned, the layering of CS is in the form of a coating which covers an entire area of the substrate or build plate per each layer throughout the entirety of the print. The CS coating is used to follow the geometric layout of the designed (desired or selected) shape, only placing material where it is needed, thus reducing the amount of material used for the print per each layer for the entirety of the print. Also, in certain embodiments, a combination of each method as defined, or a hybrid approach, is used to install more material where needed that changes on each layer or changes during the same layer. The parameters of the CS process are changed during the print as needed to contribute to collapsing any pores or voids within the print. Due to the peening process of the CS material addition, in certain embodiments, the high-velocity particles are used to micro-obliterate the voids/close pores to increase the strength as well as fatigue resistance and endurance of the final structure.

According to Step 1 of the SCSLS process, upon CS material addition, the orientation of the spray nozzle is changed from the central axis of the nozzle being perpendicular from the face of the active working plane, or face of the active substrate/build plate. Further, upon CS material addition, in certain embodiments, the particles are blocked from impacting the substrate/build plate while the heated gas stream is left on to aid in the building process (relieving in-built residual stresses, controlling thermal growth, softening different areas of the printed object, spot annealing, and the like).

In Step 2 of the SCSLS process, as aforementioned, the parameters of the laser are set to have the method of material fusion or bonding without melting but enabling migration or diffusion of atoms to be categorized as sintering. The parameters of the laser are also set to have the method of material fusion by melting or liquefaction to be categorized as welding or spot melting. The parameters of the laser are set to have the laser perform a rastering and/or vector travel with or without overlapping. Furthermore, the intensity of the laser is set, changed, or throttled depending on the location of its interface as it pertains to certain features found within the designed object, based on laser interaction related to previous interactions within the same location, or depending on the type of material used to create the structure/layer. The spot size of the laser and other related setting (speed, intensity, frequency, angle of incident, focal point, and the like) are dynamic in value and change during the entire print, certain locations of the print, and certain location of the layer. The lasers are used to scan the CS material with intent to induce heat, relax stresses, etch the surface, and modify the microstructure while not performing any particle fusion with any melting.

In Step 3 of the SCSLS process, as aforementioned, the parameters of the laser are set to have the laser perform a rastering and/or vector travel with or without overlapping. Furthermore, the intensity of the laser is set, changed, or throttled depending on the location of its interface as it pertains to certain features found within the designed object, based on laser interaction related to previous interactions within the same location, or depending on the type of material used to create the structure/layer. The spot size of the laser and other related setting (speed, intensity, frequency, angle of incident, focal point, etc.) are dynamic in value and change during the entire print, certain locations of the print, and certain location of the layer. The laser is used to scan the CS material with intent to induce heat, relax stresses, etch the surface, or alternatively change the microstructure (amorphous, extremely fine grain (<10 nm), fine grain (10 nm-100 nm) or coarse grain (>100 nm)), while not performing any particle fusion with any melting.

In Step 3a of the SCSLS process, as aforementioned, the conventional machining/material removal parameters are set to have the cutter perform a rastering and/or vector travel with or without overlapping. Furthermore, the speed and travel rate of the cutter are set, changed, or throttled depending on the location of its interface as it pertains to certain features found within the designed object, based on laser interaction related to previous interactions within the same location, or depending on the type of material used to create the structure/layer. The conventional machining/material removal global alignment changes and does not remain perpendicular in terms of the spindle axis and the plane of the substrate/build plate. In certain embodiments, this change is dynamically made throughout the whole printing process, per each layer, or many times while operating in the same layer.

According to the present invention, the layer-by-layer AM process includes where a structure is made by CS feedstock delivery and partly laser-sintered or welded allowing the powder feedstock to gain some strength until a post-processing sintering is competed upon the entire net-shape/near-net-shape part/structure either still connected to or removed from the substrate/build plate. In certain embodiments, a layer-by-layer AM process is where the deformation of the CS particle is done at a velocity and angle to all the removal and ejection of the oxide layer from the process making laser sintering/welding much more effective on all reactive materials such as Mg, Al, Ti, etc. and alloys of these, etc. Furthermore, a cycle operates with only the high-pressure gas flow to facilitate each layer to be clean or certain locations for the print. In certain embodiments, the layer-by-layer AM process is where the addition of feedstock material via CS is performed at different velocities to coincide with different laser sintering/welding techniques and power levels to aid in creating internal pores/channels/support structures along with different surface finishes of the part and differing mechanical properties of the material. In certain embodiments, the layer-by-layer AM process is capable of switching feedstock materials during the CS material addition steps. This changing of the powders is possible at any point or plane of the structure along with having the ability to mix several powders on the same plane during the CS material deposition phase. Differing materials may be used to form composites, different alloy structures, control corrosion rates of the resulting structures, control harmonics, control dampening for use in dynamic systems, adsorb or reflect differing wavelengths of electromagnetic radiation, control the mechanical properties, or even place materials which will act differently during the laser's interaction (example: installation of size-controlled internal gas pores).

The SCSLS approach provides the ability to fragment the inherent oxide layer present on the surface of reactive metals such as Mg, Al, Ti, and alloys of these elements. The SCSLS approach allows the fragmented oxide to be dispersed within the sintered particles creating a nano to sub-micron size oxide dispersion-strengthened 3D metal structure. The SCSLS approach allows the coalescence of voids and porosity generated in the CS process eliminating any voids or porosity trapped within the intrinsic sintered structure. The SCSLS approach for metal feedstock powder results in a final 3D metal structure that is fully bonded by a metallurgical bond representative of the metal. The SCSLS approach for ceramic feedstock powder results in a final 3D ceramic structure that is fully bonded by a covalent bond representative of the covalent ceramic. The SCSLS approach for ceramic feedstock powder results in a final 3D ceramic structure that is fully bonded by an ionic bond representative of the ionically bonded ceramic. The SCSLS approach for polymer feedstock powder results in a final 3D polymer structure that is fully bonded by a polymeric bond representative of the covalently bonded polymer. The SCSLS approach results in a final 3D structure that is mechanically strong and exhibiting physicochemical properties matching the intrinsic bulk structures. The SCSLS approach for a metal, ceramic, or polymer feedstock powder is used to overlay on sacrificial support structures made of ceramic, polymer, and non-reactive material that is then removed to generate engineered porous 3D structures. The CS conformed structures of metal, ceramic, or polymer are made from metal, ceramic, or polymer powder feedstock or pre-formed construct inclusive of metal injection molding (MIM) processes. The SCSLS approach is used to overlay on sacrificial support structures made of ceramic, polymer, and non-reactive materials made by MIM. The SCSLS approach is used to overlay on sacrificial support structures made of ceramic, polymer, and non-reactive materials made by MIM followed by removal of the underlying support structures to create engineered porous structures with defined porosity.

In certain embodiments, CS is used to generate thick magnesium (Mg) deposits followed by separating these thick structures of Mg from the underlying substrate and then subjecting the cold sprayed structure to sintering conditions to generate mechanically strong structures. In certain embodiments, sintering is also conducted on the cold sprayed structures to generate sintered structures. According to the invention, the SCSLS process is performed on titanium (Ti) structures. Following CS of titanium exposing the cold sprayed titanium to selective laser treatment shows that the sintering of the cold sprayed titanium layer clearly demonstrates the benefit of SLS treatment to cold sprayed metal resulting in a structure with significant elimination of voids indicating the formation of dense structures. The following benefits are realized by the SCSLS approach as opposed to performing a sintering step after the entire structure has been created by cold spray.

Fixed Build-up Layer: Inherent bond strength of the CS deposition fixes the powder to the build plate prior to laser fusion. Generation of CS powders at variable speeds also enable powder bed compaction without inducing particle deformation affording particle contact enabling also fusion of the particles during the subsequent laser melting step enabling operation particularly under microgravity and low earth orbit conditions. The process will work extremely well for certain non-reactive metals including ceramics and especially for polymeric systems. Thus, SCSLS creates vital medical, transportation, electronic, military, aerospace, marine and other parts of any configuration on earth or microgravity at any location on nonstationary and inconsistent platforms with any environment of gas or fluid with ability to operate even in under water environments with powder-bed based AM process resolutions. The process also obviates the need for stationary powder bed/build platform with limited translational (x, y, z) and rotational (0) movement including the desire for complex methods to replace the gravitational force needed to steady the powder-bed as in conventional AM processes.

Six-degrees of Freedom: The flexibility of SCSLS allows it to be placed on a robotic arm providing additional functionality in many environments. The SM phase of the SCSLS process may be, however, limited to only laser cutting depending on size and robustness of robotic arms, likely solved by alteration.

Multi-functional: The step of CS is a main feature within the SCSLS process which is a trusted standalone coating process for cladding dissimilar metals, sealing pressure vessels, and repairing structural members. Thus, having the SCSLS apparatus offers the flexibility to utilize just the CS process providing a larger range of applications from a singular unit.

Economic: The feedstock powder is kept within a hopper and is thus, protected from any unintentional laser interactions. By contrast, in current AM powder bed processes, the unneeded powder is still placed around the laser interaction area and oftentimes captures the partly melted droplets (splatters) or fine mist of spatters which are randomly ejected from the melt pool giving rise to contamination if mixed with the feedstock powders. These ejected particles when mixed in with the feed stock could also prevent the feedstock material from being reused increasing the amount of feedstock material needed for a series of uses.

The invention includes but is not limited to the following applications.

Fabrication of heat transfer devices for thermal energy conversion applications and thermal management. High-temperature heat pipes are usually employed as passive heat transfer technologies due to their high efficiency for high-temperature heat exchangers, nuclear and solar reactors, space reactors, and solar energy storage uses with phase change materials. Alkali metals (e.g., sodium and lithium) and silver are typically used as working fluids for high-temperature heat pipes needing the tube and casing materials to be corrosion resistant related to working fluids and vapors. Tantalum (Ta), tungsten (W), molybdenum (Mo), and niobium (Nb) that are mutually soluble and meet specifications as described in Table 1 are preferred for use with sodium, lithium, and silver as working fluids. Integration of the proposed SCSLS process with heat pipes, heat exchangers, and heat sinks, is useful to fabricate customized and complex freeform parts with varying degree of porosity and micro-channels.

Functionally gradually generated gradient materials for the spacecraft wing leading edge. The conventional process uses about an inch of tungsten plate for the spacecraft leading edge which is then attached to some other metal (e.g., stainless steel) behind it. Two dissimilar metals attached in this way can introduce significant performance issues at high temperatures due to their different properties. This is a major design limitation and will impact the overall capabilities of a spacecraft. Hence, developing a process capable of building a leading edge starting with refractory alloys and then slowly grading the composition into another light-weight metal with clean structural connections offers great benefits. The proposed SCSLS process enables simultaneous or sequential multi-metals delivery with consequent sintering forming strong metallic bonds. Thus, a tailored use and design-specific compositional anisotropic metal composites for leading edge technology can be engineered in a single process. Much savings in energy, manufacturing, and process management costs can also be envisaged.

Advanced high-heat-flux and plasma-facing materials. Tungsten and copper alloys, and W—Ag and W—Ag—Cu composites, are preferred plasma facing and heat sink candidate materials, respectively when utilized in Plasma Facing Components (PFCs) of fusion reactors. However, large melting point differences and variation in coefficient of thermal expansion of W and Cu limits PFCs fabrication by conventional methods and hence, use of functionally graded materials (FGMs) of W and Cu has great value. FGMs are basically two-phase particulate composites synthesized such that the volume fractions of the constituents vary continuously in the thickness direction to give a predetermined composition profile. The SCSLS approach is useful to easily fabricate these FGMs with varying compositions and porosities by using a multiple nozzles-based CS system or adjusting the metal powder feed from multiple metal powder hoppers. Further, in future nuclear fusion reactors such as tokamaks or stellarator reactors, the divertor and inner-most chambers are expected to be made of tungsten and coated with non-porous dense coatings of self-passivating tungsten base alloys of W-chromium (Cr)-yttrium (Y) and W—Cr—Ti. The SCSLS process is useful to make dense W coatings with excellent interface qualities with self-passivating W-based alloys.

FIG. 1 is a schematic that shows the steps of a process for the additive manufacturing method of the SCSLS process, according to certain embodiments of the invention. Step 1 shows material placed in semi-precise locations utilizing CS deposition. The CS process collides particles onto a substrate transferring kinetic energy created by a heated, high-pressure gas stream traveling at a critical velocity. The particles on collision endure severe plastic deformation creating crevice interlocking and adiabatic shear instabilities mimicking localized cold welding events, firmly adhering the feedstock to the structure. Fracturing of the surface layer comprised of oxides and other impurities caused by severe plastic deformation of particles and removal of these fragments from the local area is caused by the turbulent gas streams used to accelerate the particle, thus creating clean surfaces. New oxide formation on the surface of high-oxygen-affinity particles is prevented due to the tight particle-particle CS induced interlocking of particles. Intrinsic coating strength allows each particle to maintain and remain in position, thus resisting the turbulent gas flows used during additional passes of cold spray (CS) deposition. The installation of a compressive stress within the coating helps to mitigate the formation of tensile stresses placed into the structure during the thermal phase. Step 2 shows that the dense CS coating is exposed to a material-specific localized laser sintering (LS) or laser melting (LM) enabling the CS deposited particles in the previous step to be coalesced and fused. In certain embodiments, for SCSLS of titanium (Ti), LM enables fusion, while inter-atomic diffusion induced by LS supports SCSLS of magnesium (Mg). Both mechanisms favor making dense W parts since W can be sintered or melted, thus also enabling parts of W-tantalum (W—Ta), tungsten niobium, tungsten cobalt, and tungsten rhenium alloys, and W-copper (W—Cu), tungsten-silver, tungsten-tungsten carbide, and W—Rh composites. The LS step of the SCSLS process provides high resolution because the laser facilitates the localized precision particle fusion event, thus mimicking the high levels of resolution currently provided by conventional selective laser melting (SLM) and selective laser sintering (SLS) based AM processes; and reproducibility because the surface tension of the melt pool is prevented from drawing in nearby particles since the deposited feedstock material is retained into position prior to interaction with the laser, whereby prevention of this unpredictable event allows a more consistent melt pool to be generated providing for creation of structures with a reproducible set of materials properties; as well as reduced energy demand because the high surface contact and weak metallurgical bonds characteristic of CS particles and layers allow for more consistent heat dissipation throughout the build, thus mitigating any localized areas of high thermal stresses to be built up while facilitating a uniform cooling rate of the melt pool and furthermore, the reduced energy requirement due to the creation of close particle-particle contact needed to perform the particle fusion event also decreases material evaporation caused by the extreme environments generated within the laser spot. Step 3 shows SM, which is executed either by conventional milling or precision laser cutting resulting in a smooth finish of both the outer surface and any internal features. The SM step provides corrosion protection because the precision smooth surface finish of SM aids in creating corrosion resistant parts by removing corrosion causing crevices that hold corrosive materials stationary against the surface; and improved laminar fluid flow due to the ability to create smooth internal passageways reduces the need for larger-diameter flow networks to compensate for wall friction enacted on the flow of the fluid, thus allowing for the creation of efficient laminar flow within a smaller conduit; and minimization of surface defects because the precision SM step also allows for leveling of the working surface, thus ensuring high precision builds to be created in the event of balling or other laser melting anomalies commonly encountered in SLM-based AM processes; as well as incorporation of non-AM surface features due to the ability to create features such as threaded blind holes and thin non-porous partitions which are cumbersome to create with other AM methods while maintaining a level of high precision along with creating a smooth surface finish. After completion of the SM portion of the SCSLS process, one layer is effectively completed. The start of the second layer thus returns the procedure back to Step 1, i.e., the CS material deposition phase. Since the feedstock particles are deposited under high velocity having a large amount of kinetic energy, the particle upon collision with the previous layer can annihilate any pores, cracks, or other unintentional voids located within that layer. These closed voids and cracks will then be sealed during further interaction with the laser in the subsequent step hence, enabling the creation of near-net-shaped, fully dense solid structures.

### EXAMPLES

Various experiments were conducted and structures prepared to test and evaluate the novel SCSLS process. During the CS of Mg and Al, the postulate of oxide removal and ejection from the process was validated along with evidence of individual particle coalescing during bulk sintering. Experiments were also conducted using cold sprayed Mg powder within the SLS stage wherein the precision laser sintering showed promise once the processing parameters were optimized for the material. The parameters of the laser for vaporization of the unused CS deposited Mg material were also identified, rendering satisfactory results in shaping and removing any non-sintered material.

In general, CS deposition was achieved using magnesium, aluminum, iron, copper, and Inconel as the primary material with satisfactory mechanical interlocking and bonding utilizing two differing accelerating gases (e.g., nitrogen and helium). Post-process annealing and sintering were completed with internal features being formed at defined time/temperature holds and differing process procedures within a tube furnace providing post SCSLS processing knowledge if needed to finalize a structure. High-intensity laser cutting, material vaporizing, and material shaping were successfully attempted with studies to quantify and optimize the laser when interacting with CS Mg samples contacting several layers of thickness to mimic the SCSLS process. Laser sintering of CS deposited Mg was also successfully demonstrated with work initiated to optimize the parameters for translation into the SCSLS process. Further, there was demonstrated the strength of the bonded CS layering deposit and its ability to maintain visible integrity of the CS construct while inverted, struck, or exposed to vibration before the interaction of the laser with the material as it welds the particles into a strong solid structure.

### Example 1

### CS Sample Post Spraying Before any Thermal/Sinter Process

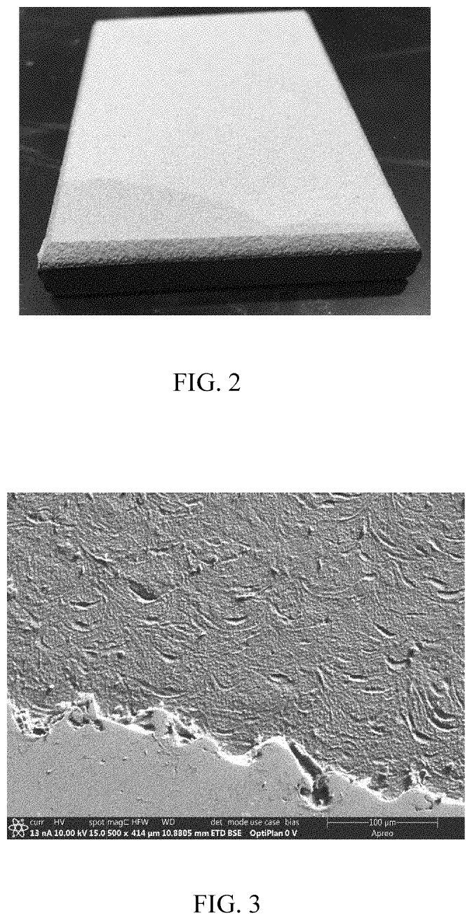

Samples were created with cold spray (CS) magnesium (Mg) deposition as shown in FIG. 2, which depicts the coating created by accelerating Mg particles to a high velocity and allowing them to collide with a substrate. Under a scanning electron microscope (SEM), the micrograph shows the high levels of deformation each particle undergoes shown as arced lines (see FIG. 3) indicating the deformed zones of the particles. This bonding is mechanical in nature with high corrosion rates and brittleness discovered under testing due to the many surface areas and voids caused by this type of bonding. Thermal post processing was conducted to induce sintering to control the corrosion rates and mechanical properties of the sample. It was discovered with the use of different thermal cycles, the material's properties could be selected over a large range of available parameters.

### Example 2

### Controlled Mechanical Strength Adjustment

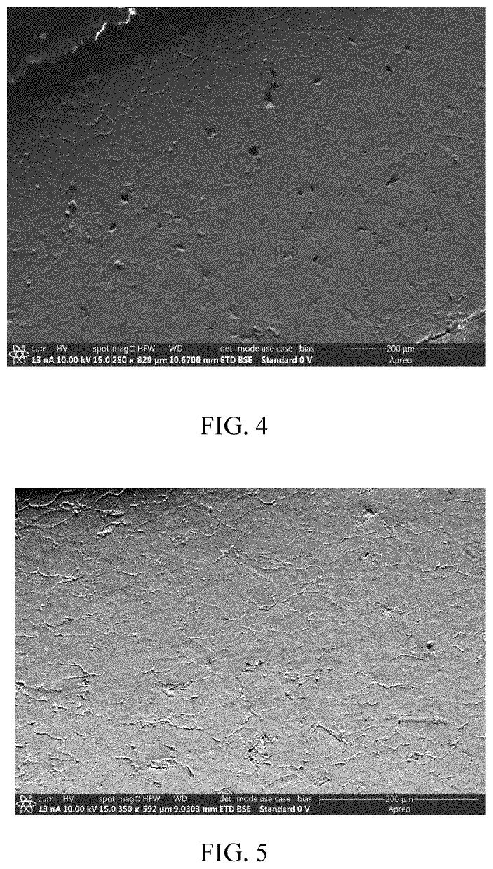

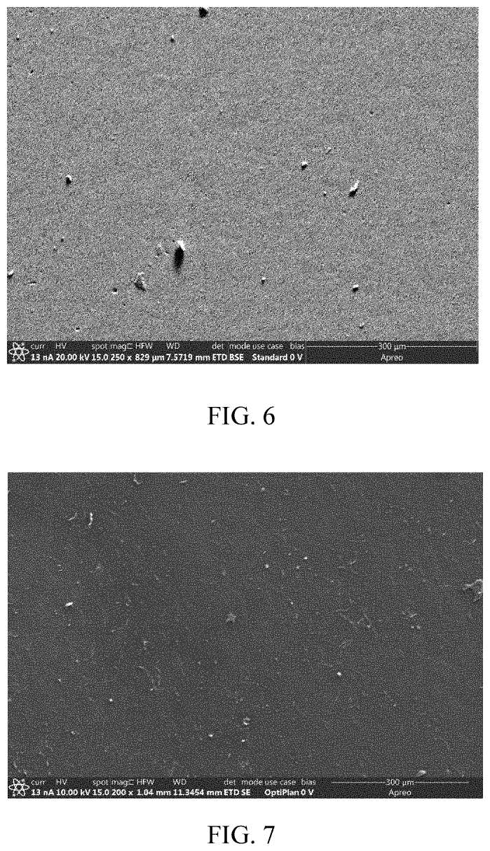

A sample having a similar cross section as viewed in FIG. 3, was subjected to a selection of different thermal processes differing in the amount and range of the thermal cycling to reduce the strength of the overall structure by removing a portion of the mechanical bonding without the creation of any metallic bonding (see FIG. 4). Utilizing another set of thermal cycling parameters, the mechanical bonding was reduced and replaced with the formation of a metallic bond allowing the ductility to increase and resulting in a reduction in internal particle boundaries (see FIG. 5). Further, under yet another set of thermal cycling parameters all of the mechanical bonding was replaced with metallic bonding allowing an isotropic structure with high levels of ductility and the elimination of internal particle boundaries (see FIG. 6). The list of parameters used to create each sample is included in Table 2 below.

TABLE 2
Listing of parameters used to define
level of mechanical/metallic bonding

| | | | | | | | Initial |
|---|---|---|---|---|---|---|---|
| | | | | | | | Annealing |
| | Mg | CS | CS | | High | Total | (Temp./ |
| | Feed | Press. | Temp. | No. of | Temp. | Time | Time) |
| Sample | (rpm) | (psi) | (° C.) | Cycles | (° C.) | (hr) | (° C./hr) |
| FIG. 4 | 1 | 500 | 600 | 2 | 500 | 88 | No |
| FIG. 5 | 1 | 500 | 400 | 4 | 500 | 48 | 200 |
| FIG. 6 | 3 | 800 | 400 | 4 | 500 | 130 | 200 |

### Example 3

### Thermal Pore/Channel Processing

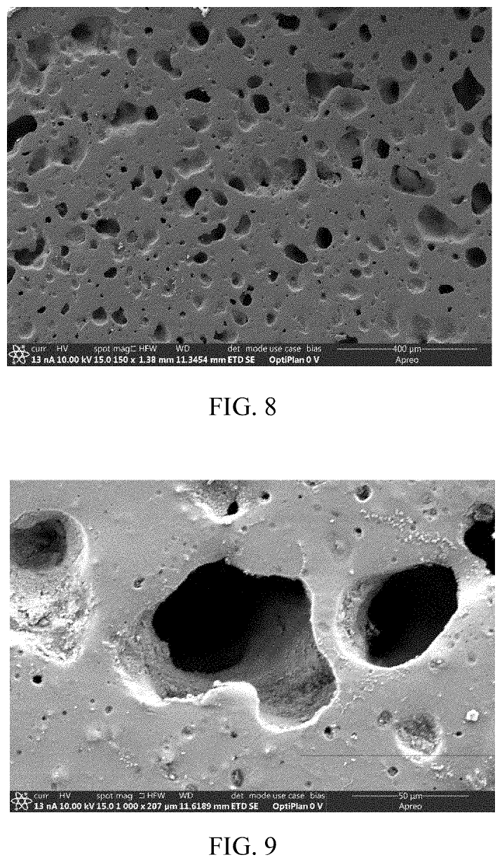

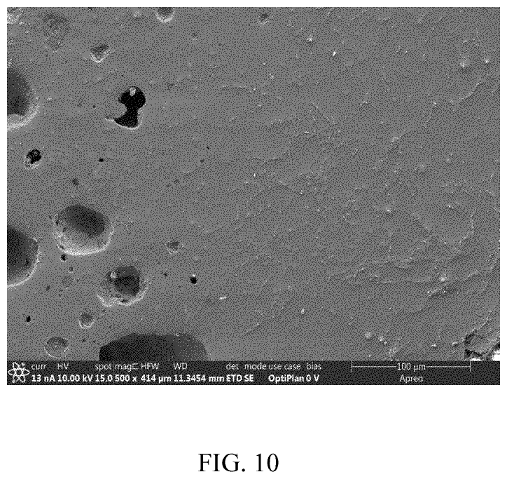

Using the CS Mg deposition process parameters along with the thermal cycling parameters, it is possible to generate desired pore and channels as needed. The micrograph shown in FIG. 7 is a sample with relatively no pores or channels and is mostly metallically bonded. Changing the parameter through most of the process however, gains a cross section similar to FIG. 8 having both closed pores and open interconnected channels. FIG. 9 shows a magnification of the interconnected channels with large (30 μm) and small (0.5 μm) spherical pores surrounding it. These channels vary in lengths and many do exit the structure while the diameter ranges between 30-70 μm. For biomedical applications, these channels are critical since they allow fluid movement and migration of cells and nutrients once the cells attach internally to the structure. FIG. 10 shows it is also possible to restrict these pores to certain areas of the structure allowing anisotropic material properties with varying densities located in different areas of the same structure. Table 3 contains an overview of the differing parameters used to create these voids within the samples listed.

TABLE 3
Listing of parameters used to define
level of mechanical/metallic bonding

| | | | | | | | Initial |
|---|---|---|---|---|---|---|---|
| | | | | | | | Annealing |
| | Mg | CS | CS | | High | Total | (Temp./ |
| | Feed | Press. | Temp. | No. of | Temp. | Time | Time) |
| Sample | (rpm) | (psi) | (° C.) | Cycles | (° C.) | (hr) | (° C./hr) |
| FIG. 7 | 1 | 500 | 400 | 4 | 500 | 48 | 200 |
| FIG. 8 | 3 | 800 | 400 | 4 | 600 | 130 | No |
| FIG. 9 | 3 | 800 | 400 | 4 | 600 | 88 | No |

### Example 4

### Energy Dispersive X-Ray Spectroscopy

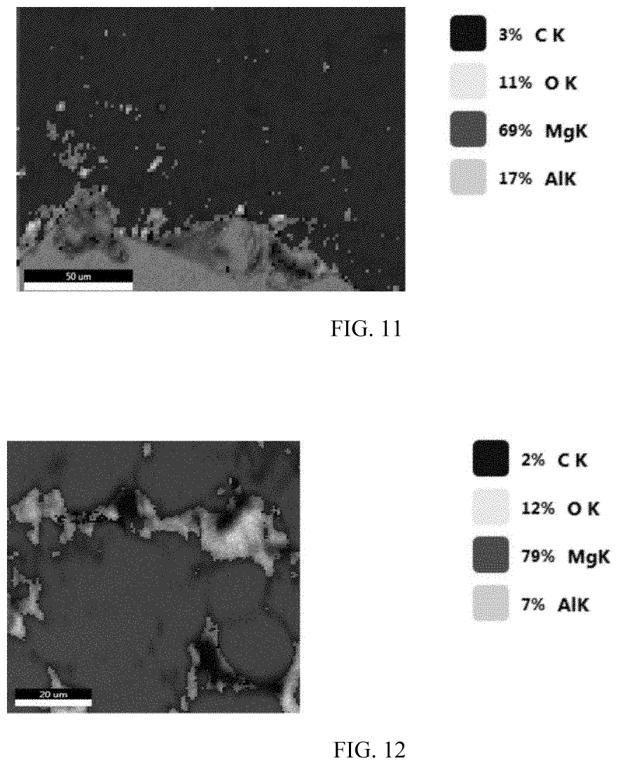

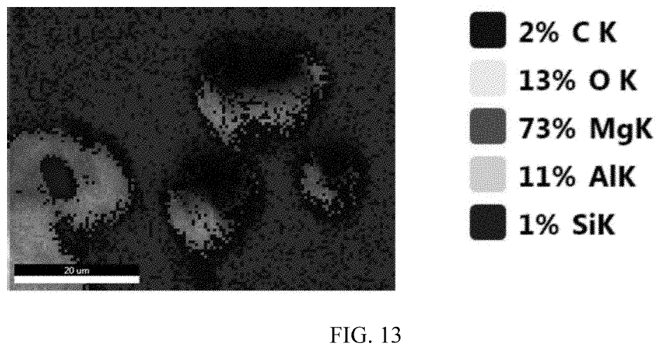

Following the distribution of the oxygen content, shows why post CS sintering is possible using reactive metals such as Mg. FIG. 11 is post-CS deposition and pre-thermal processing highlighting the Mg particles are not surrounded by the white region depicting oxygen-containing compounds as shown in FIG. 12. As stated above, this allows sintering to metalize the mechanical bonding and correspondingly, change the material's properties. This mechanical bond caused by the CS deposition process can be relaxed causing particle adhesion to diminish, thus the metal-to-metal contact is lost and sintering is no longer possible. This also allows the oxides, the oxygen content varying depending on the powder feed characteristics but likely to be in the 10-15% range for particle size in the 30-100 micron size range, to form in the voids around the particles ensuring that the void remains (see FIG. 12). Having control and understanding over this system allows a wider range of mechanical properties to be achieved and located within the sample from the same type of material. Finally, in the case of the pores and channels, oxides again form within these voids, but the base material shows no signs of particle boundaries allowing many degrees of sintering to be achieved (see FIG. 13). This analysis supports the claim of having a wide range of adjustable material properties depending on the amount of mechanical versus metallic bonding which can not only be used to modify a material to encompass but also achieve a wide range of optimized values. This analysis also shows that these same procedures can be used to install predicable and designed internal features within a CS coating process allowing it to be transformed into a method of additive manufacturing to generate materials with unique properties such as those possible from Mg, including refractory metals such as W, Ta, Mo, and their alloys as well as other metals such as Ti, Al and their alloys, including non-reactive metallic systems such as Cu, Fe, and steel, and their alloys.

1. A method of preparing a multi-layer structure, comprising:
a) cold spraying a layer of feedstock material onto a substrate/build plate to create a cold sprayed coating;
b) employing an energy source to sinter or melt the cold sprayed coating, comprising:
directing the energy source to conduct localized sintering or melting of specific particles or regions of the cold sprayed coating that correspond to a preselected multi-layer structure; and
forming a sintered or melted layer; and
c) sequentially repeating the steps of a) and b) forming one or more additional material layers on the first material layer,
wherein the number of additional layers is determined based on a total number of layers needed to produce the preselected multi-layer structure.

2. (canceled)

3. (canceled)

4. The method of claim 1, wherein the feedstock material is in a dry form selected from the group consisting of particles, particulates, or granules.

5. The method of claim 1, wherein the sintering or melting process results in a fragmented oxide dispersed within the sintered or melted material and the preselected multi-layer structure.

6. (canceled)

7. The method of claim 1, wherein adding a second material layer to the first material layer is effective to control or eliminate porosity in the first material layer.

8. (canceled)

9. The method of claim 1, wherein following completion of the first material layer and a subsequent second material layer, power of the energy source is increased and used in trimming and shaping an outline of the preselected structure being prepared.

10. (canceled)

11. The method of claim 1, wherein each of the feedstock materials comprises a material selected from the group consisting of metal, metal alloy, polymer, ceramic, semiconductor, and mixtures and combinations thereof.

12. (canceled)

13. The method of claim 1, further comprising employing a CAD model corresponding to the preselected structure for directing and controlling the energy source in step b).

14. The method of claim 1, wherein step b) forms pores and/or pathways in the preselected multi-layer structure.

15. The method of claim 1, further comprising following step b) subjecting the sintered or melted layer to subtractive machining, comprising:
smoothing the surface of the sintered or melted layer; and
forming a first material layer of the preselected multi-layer structure.

16. (canceled)

17. An additive manufacture device, comprising:
a cold spray apparatus to deposit a coating of feedstock material on a substrate/build plate;
an energy source to sinter or melt the coating of feedstock material to form a sintered or melted layer; and
a CAD model to direct the energy source to sinter specific particles or regions of the coating of the feedstock material that correspond to a preselected multi-layer structure,
wherein one or more additional layers of feedstock material are sequentially deposited on the first material layer to produce one or more additional material layers, and wherein the number of additional material layers is determined based on those needed to produce the preselected multi-layer structure.

18. An additive manufacture device, comprising:
a cold spray apparatus to deposit compact powder of feedstock material on a powder bed generating a compact powder bed;
an energy source to sinter or melt the compact powder bed of feedstock material to form a sintered or melted layer; and
a CAD model to direct the energy source to sinter specific particles or regions of the compact powder layer of the feedstock material that correspond to a preselected multi-layer structure,
wherein one or more additional layers of feedstock material are sequentially deposited on the first material layer to produce one or more additional material layers, and wherein the number of additional material layers is determined based on those needed to produce the preselected multi-layer structure.

19. The device of claim 17, wherein the feedstock material comprises a material selected from the group consisting of metal, metal alloy, polymer, ceramic, semiconductor, and mixtures and combinations thereof.

20. (canceled)

21. The device of claim 17, wherein the multi-layer structure is a medical implant device.

22. The device of claim 18, wherein the feedstock material comprises a material selected from the group consisting of metal, metal alloy, polymer, ceramic, semiconductor and mixtures and combinations thereof.

23. (canceled)

24. The device of claim 18, wherein the multi-layer structure is a medical implant device.

25. (canceled)

26. (canceled)

27. (canceled)

28. The device of claim 17, wherein the one or more of the additional material layers of the multi-layer structure is/are composed of a different feedstock material as compared to the other material layers.

29. (canceled)

30. (canceled)

31. The device of claim 17, further comprising a subtractive machining apparatus to smooth the surface of the sintered or melted layer and form a first material layer of the preselected multi-layer structure.

32. (canceled)

33. The device of claim 18, wherein the one or more of the additional material layers of the multi-layer structure is/are composed of a different feedstock material as compared to the other material layers.

34. (canceled)

35. The device of claim 18, wherein the feedstock material is selected such that alternating material layers are composed of the same feedstock material or a pattern of material layers are composed of the same feedstock material.

36. The device of claim 18, further comprising a subtractive machining apparatus to smooth the surface of the sintered or melted layer and form a first material layer of the preselected multi-layer structure.
